SEQUENCE LISTING (1 sequence)

SEQ ID NO: 1 - DNA, 1792 bp, phaC Aeromonas hydrophla 4 AK 4
  misc_feature (490)..(490): n is a, c, g or t
  misc_feature (625)..(625): n is a, c, g, or t
  misc_feature (872)..(872): n is a, c, g or t
  misc_feature (930)..(930): n is a, c, g or t
  misc_feature (1096)..(1096): n is a, c, g or t
  misc_feature (1167)..(1167): n is a, c, g, or t
  misc_feature (1175)..(1175): n is a, c, g or t
  misc_feature (1181)..(1181): n is a, c, g or t
  misc_feature (1195)..(1195): n is a, c, g or t
  misc_feature (1198)..(1198): n is a, c, g or t
  misc_feature (1204)..(1204): n is a, c, g or t
  misc_feature (1205)..(1205): n is a, c, g, or t
  misc_feature (1207)..(1207): n is a, c, g or t
  misc_feature (1219)..(1219): n is a, c, g or t
  misc_feature (1220)..(1220): n is a, c, g, or t
  misc_feature (1227)..(1227): n is a, c, g or t
  misc_feature (1252)..(1252): n is a, c, g or t
  misc_feature (1255)..(1255): n is a, c, g or t
  misc_feature (1260)..(1261): n is a, c, g or t
  misc_feature (1283)..(1283): n is a, c, g or t
  misc_feature (1299)..(1299): n is a, c, g or t
  misc_feature (1320)..(1320): n is a, c, g or t
  misc_feature (1324)..(1324): n is a, c, g or t
  misc_feature (1325)..(1325): n is a, c, g, or t
  misc_feature (1343)..(1343): n is a, c, g or t
  misc_feature (1422)..(1422): n is a, c, g or t
  misc_feature (1485)..(1485): n is a, c, g or t
  misc_feature (1579)..(1579): n is a, c, g or t
  misc_feature (1607)..(1607): n is a, c, g, or t
  misc_feature (1769)..(1769): n is a, c, g or t
atgagccaaccatcttatggcccgctgttcgaggccctggcccactacaatgacaagctgctggccatggccaaggcccagacagagcgcaccgcccaggcgctgctgcagaccaatctggacgatctgggccaggtgctggagcagggcagccagcagccctggcagctgatccaggcccagatgaactggtggcaggatcagctcaagctgatgcagcacaccctgctgaaaagcgcaggccagcagagcgagccggtgatcaccccggagcgcagcgatcgccgcttcaaggccgaggcctggagcgaacaacccatctatgactacctcaagcagtcctacctgctcaccgccaggcacctgctggcctcggtggatgccctggacggcgtcccccagaagagccgggagcggctgcgtttcttcacccgtcagtacgtcaacgccatggcacccagcaacttcctggccaccaacccggagctgntcaagctcaccctggagtccgacggccagaacctggtgcgcgggctggccctcttggccgaggatctggagcgcagcgccgatcagctcaacatccgcctgaccgacgaatccgccttcgagctcggccgggatntggcgaccaccccgggccgggtggtgcagcgcaccgagctctatgagctgatccagtacagccccaccacggaaaccgtgggcaagacgcctgtgctgatcgtgccccccttcatcaacaagttctacatcatggacatgcggccccagaactccctggtcgcctggctggtcgcccagggccagacggtgttcatgatctcctggcgcaacccgagcgtagcccaggcccaaatcgatctcgacgnctacgtggtggatggcgtcatcgccgccctggacggcgtggaagcggccaccggcgancgggaggtgcacggcatcggctactgcatcggcggcaccgccctgtcgctcgccatgggctggctggcggcgcggcgccagaagcagcgggtgcgcactgccaccctgttcaccaccctgctggacttctcccagccagggggagcttggcatcttcattcacgancccatcatatcggcgctcgaagcgcaaaatgaagccaagggcatcatggacgggcgccagcgtggtttttntttcagcntggggngggagaacagcttntantggaannanaacatggacagnnacttcanggtcagagcccggtggctttagatntgntgcannggaacagcgacagcaccaatgnggcgggcaagacccanaacagcgtggtgcgctgtttntatnnggagaaccagctggtgangggggagttcaagatccgcaacacccgcatcgatcttggcaaggtgaagacccctgtgctgctggtgtcggcggtggangatcacatcgccctctggcagggcacctggcagggcatgaagctgtttggcggggagcagcgnttcctcctggcagatccggccacatcgccggcatcatcaacccgccggtcgccaacaagtacggcttctggcacaacggggccgaggccgatancccggagagctggctggcaggggcgacncatcagagcggctcctggtggcccgagatgatgggctttatccagagccgtgacgaagggtcagagcccgtccccgcacgggtgcccgaggaggggctggcccccgcccccggccactatgtcaaggtgcggctcaaccccgtgtttgccagcgccacagangatgacgccgcatgagcgcacaa

(19) United States
(12) Patent Application Publication
(10) Pub. No.: US 2007/0161097 A1
(43) Pub. Date: Jul. 12, 2007

(54) Deregulated bacteria with improved polyhydroxyalkanoate production

(75) Inventors: Phillip Richard Green, Wyoming, OH (US); Robert Ian Johnson, Cramlington (GB); Stuart Antony Dunn, Cramlington (GB)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(73) Assignee: The Procter & Gamble Company

(21) Appl. No.: 11/517,979

(22) Filed: Sep. 8, 2006

Related U.S. Application Data
(60) Provisional application No. 60/715,028, filed on Sep. 8, 2005.

(30) Foreign Application Priority Data
Feb. 28, 2005 (GB) PCT/GB05/00739

(51) Int. Cl. C12P 7/62; C12N 1/21

(52) U.S. Cl. 435/135; 435/252.3

(57) ABSTRACT

Mutant bacteria with improved poly-3-hydroxylalkanoate (PHA)-producing characteristics and methods of producing and using them are provided.

### CROSS REFERENCE TO RELATED APPLICATION

This Application claims priority to U.S. Provisional Application Ser. No. 60/715,028, filed Sep. 8, 2005 (P&G Case 9993P).

### FIELD OF THE INVENTION

The invention relates to mutated microorganisms that constitutively produce elevated levels of polyhydroxyalkanoate (PHA) biopolymer.

### BACKGROUND OF THE INVENTION

The ability of numerous microorganisms to synthesize and accumulate a polymer of β-hydroxyalkanoic acid (polyhydroxyalkanoate, PHA) as an energy storage compound has long been recognized. The most commonly found compound of this class is poly(D(−)-3-hydroxybutyrate) (PHB). However, some microbial species accumulate copolymers, which in addition to hydroxybutyrate, may contain longer chain hydroxyalkanoates. Interest has focused on these PHAs because these biopolymers are thermoplastics that are biocompatible, biodegradable and exhibit physical properties resembling the properties of petro-chemically-based polymers such as polyethylene and polypropylene.

One exemplary bacteria that produces PHA, Wautersia eutropha (also known as Ralstonia eutropha or Alcaligenes eutrophus), accumulates PHA during fermentation in response to limitation of critical nutrients such as phosphate or nitrogen. PHA is typically produced by fermentation of bacteria in two stages. In the first stage, the bacteria are cultivated in media that contains a full supply of nutrients permitting multiplicative growth. The bacteria are allowed to multiply until it reaches sufficient biomass, usually measured as dry cell weight per liter. In the second stage, the availability of at least one nutrient that is required for growth is restricted, e.g., nitrogen or phosphorus, which has the effect of limiting cell division and inducing PHA production. Significant PHA production does not occur until the second stage, when the nutrient limitation induces accumulation of PHA.

Most PHA-producing organisms do not produce significant levels of PHA under non-limiting growth conditions. For example, Wautersia eutropha (Ralstonia eutropha) has been reported to produce only 3% PHA as a percentage of dry cell weight when cultured under conditions in which no nutrients are limited. Repaske et al., Appl Environ Microbiol. 32: 585-591, 1976.

There exists a need for modified bacteria that produce significant levels of PHA in the presence of non-limiting levels of nutrients.

### SUMMARY OF THE INVENTION

The present invention provides isolated nutrient-deregulated modified bacteria that produce surprising amounts of PHA, compared to unmodified bacteria, in the presence of media containing a sufficient amount of nutrients to permit multiplicative growth. Such bacteria produce significant levels of PHA in the presence of non-limiting levels of important nutrients such as nitrogen, phosphorus, magnesium, sulfate and potassium.

The novel isolated bacteria of the invention are capable of (a) producing at least 10% polyhydroxyalkanoate (PHA) by dry cell weight under culture conditions in which levels of nutrients are not limited and (b) producing at least 20% PHA by dry cell weight under culture conditions in which iron is limited but no other nutrients are significantly limited. The invention optionally excludes isolated bacteria previously known in the art that produce about 50% or more PHA by dry cell weight under culture conditions in which levels of nutrients are not limited, and that exhibit at least about a 10% increase in PHA production under culture conditions in which iron is limited but no other nutrients are significantly limited.

Exemplary bacteria are selected from Ralstonia species, Alcaligenes species, Wautersia species, Zoogloea species, Bacillus species, Aeromonas species, Azotobacter species, Clostridum species, Nocardia species, Halobacterium species, or Pseudomonas species, or combinations thereof. The bacteria of the invention specifically exclude bacteria known to produce over 50% PHA by dry cell weight under culture conditions in which levels of nutrients are not limited, such as Alcaligenes latus and an Azotobacter vinlandii mutant (mutation in NADH oxidase). Alcaligenes latus produces PHB at approximately 88.3% of the weight of the cell (Lee et al., Polym. Degradation Stab. 59: 387-393, 1998) and Azotobacter vinlandii produces PHA at approximately 94% of the weight of the cell (Page et al., Can. J. Microbiol. 41: 106-114, 1995).

Such modified bacteria may deregulated for one nutrient, e.g., either phosphate-deregulated or nitrogen-deregulated, or may be deregulated for two or more nutrients, e.g. both phosphate-deregulated and nitrogen-deregulated. Preferably, the bacteria are nutrient-deregulated PHA-producing organisms, or PHA-negative mutants that are additionally engineered to contain a non-native PHA-producing gene, e.g. phaC, A and/or B.

Exemplary bacteria are those deposited on Jun. 1, 2005 with the American Type Culture Collection (ATCC), P.O. Box 1549 Manassas, Va. 20108, USA, under Accession Nos. PTA-6759 and PTA-6760, respectively.

The invention also provides isolated bacteria that exhibit all of the identifying characteristics of the bacteria deposited under ATCC Accession No. PTA-6759 or the bacteria deposited under ATCC Accession No. PTA-6760. Exemplary embodiments of such bacteria are descendants of the deposited bacteria, or mutants of the deposited bacteria, that retain the identifying characteristics of the deposited bacteria, i.e., that retain the same or similar mutations that cause increased PHA production, compared to unmodified organisms, under culture conditions in which levels of nutrients are not limited.

In addition, the invention provides cultures containing the isolated bacteria of the invention and a culture medium. The invention further provides methods of producing PHA using any bacteria of the invention described herein. Such production methods comprise the step of growing the bacteria in suitable culture media so that the bacteria produce PHA. Further optional steps include harvesting the bacteria, and/or extracting PHA from the bacteria. Alternatively, if the PHA is secreted into the culture media, the methods may comprise steps of growing the bacteria in suitable culture media and extracting PHA from the culture media.

Any culture media and culture methods known in the art may be used as long as sufficient nutrients are supplied to the organisms to permit growth or production of PHA. In one aspect, the invention thus provides methods of culturing such bacteria in media that is not limited in an essential nutrient, i.e. nutrients are present at a concentration such that they are never limiting to the extent causing cessation of multiplicative growth. By way of example, the cultivation process includes transferring the bacteria from one fermentor to another (scaling up), as well as batchwise or continuous feeding, provided that nutrients in the culture media are not limited to a concentration that would significantly impair multiplicative growth.

In another aspect, the invention further provides methods of culturing bacteria of the invention according to two-step cultivation methods in which the bacteria undergo a second step of cultivation in culture media that is limited in one or more essential nutrients, e.g. inorganic elements.

The invention also provides improved methods of producing PHA by limiting trace elements for which the bacteria has not been deregulated. For example, after a sufficient time period of multiplicative growth, enhanced production of PHA may be achieved by growing a phosphate-deregulated bacteria in culture media containing limiting levels of an element other than phosphorus, e.g. magnesium, sulfate or iron, preferably iron.

Methods are provided that produce PHA copolymers composed of C4 and medium chain length monomers (e.g. monomer units with greater than five carbons). Exemplary copolymers include copolymers containing C6, C7, C8, C10, C12, C14, C16, and C18 copolymers, e.g. 3-hydroxyhexanoate (HH) (C6), 3-hydroxyheptanoate (HHp) (C7) and/or 3-hydroxyoctanoate (HO) (C8), particularly copolymers that contain C4 and C6 (particularly 3-hydroxybutyrate and 3-hydroxyhexanoate or the corresponding acids), e.g. polyhydroxybutyrate-co-hexanoate (C4-C6), and most particularly at ranges of 80-98 mol % C4 and 2-20 mol % C6. Additional copolymers and suitable media are described in U.S. Pat. No. 6,225,438.

### DETAILED DESCRIPTION OF THE INVENTION

The present invention provides isolated, modified “nutrient-deregulated” bacteria that produce surprising amounts of PHA, compared to unmodified bacteria, in the presence of media containing a sufficient amount of nutrients to permit multiplicative growth. Such modified bacteria may be deregulated for one nutrient, e.g. either phosphate-deregulated or nitrogen-deregulated, or may be deregulated for two or more nutrients, e.g. both phosphate-deregulated and nitrogen-deregulated. Other exemplary modified bacteria include magnesium-deregulated, sulfate-deregulated, potassium-deregulated or iron-deregulated organisms.

In exemplary embodiments, the bacteria of the invention are capable of (a) producing at least 10%, 20%, 25%, or 30% or higher PHA by dry cell weight under culture conditions in which levels of nutrients are not limited and (b) producing at least 20%, 30%, 40%, 50%, 60%, 65%, 70%, 75%, or 80% or higher PHA by dry cell weight under culture conditions in which iron is limited but no other nutrients are significantly limited. Such bacteria include any bacteria known in the art that produce PHA, but specifically exclude bacteria known to produce over 50% PHA by dry cell weight under culture conditions in which levels of nutrients are not limited, such as Alcaligenes latus and an Azotobacter vinlandii mutant (mutation in NADH oxidase), which each accumulate PHB to levels of over 90% of the weight of the cell.

By producing PHA in a phosphate deregulated bacterium, PHA production occurs constitutively even in the presence of rich media that contains no limitations of important nutrients. The ability to utilize rich media or richer, inexpensive media throughout the fermentation permits faster growth of bacteria, higher production of PHA, and a more industrially robust PHA production process at a reduced cost. (See Examples 8 and 9.) Eliminating the necessity of restricting one or more nutrients also allows for continuous fermentation (i.e. eliminates the need for a two-step cultivation method).

As used herein, “isolated bacteria” refers to a population of a single strain of bacterium that has been identified and separated from a component of its natural environment. For example, bacteria that are part of a culture composition containing artificial culture medium is considered isolated.

As used herein, “culture medium containing non-limiting levels of nutrients” or “culture medium in which levels of nutrients are not limited” refers to culture medium that contains an adequate supply of all nutrients to permit bacteria to rapidly multiply. Similarly, “non-limiting level of a nutrient” refers to a concentration of that nutrient in culture medium which is adequate to support rapid multiplicative growth. Conversely, “culture medium in which a nutrient is limited” refers to culture medium in which the level of that nutrient is reduced to a concentration that causes multiplicative growth of bacteria to essentially cease, and a “limiting level of a nutrient” refers to a concentration of that nutrient in culture medium which causes multiplicative growth of bacteria to essentially cease.

As used herein, “multiplicative growth” refers to a rapid increase in numbers of bacteria such as observed during the exponential growth phase.

As used herein, a “phosphorus-deregulated” bacteria refers to a bacteria that has a defect in phosphorus or phosphate regulation such that one or more genes that is normally upregulated (or repressed) by phosphorus depletion is constitutively upregulated (or repressed), with the result that the bacteria synthesizes increased levels of PHA even in the absence of phosphorus depletion. Similarly, a “nitrogen-deregulated” bacteria refers to a bacteria that has a defect in nitrogen regulation such that one or more genes that is normally upregulated (or repressed) by nitrogen depletion is constitutively upregulated (or repressed), with the result that the bacteria synthesizes increased levels of PHA even in the absence of nitrogen depletion. The meaning of other nutrient-deregulated bacteria corresponds similarly.

As used herein, a “PHA-negative” mutant bacteria refers to a bacteria that has been mutated so that it does not produce PHB or PHA. See, e.g., Schlegel et al., Arch. Mikrobiol. 71:283-4830, 1970]. Such a bacteria may be genetically engineered to produce PHA by inserting a desired PHA gene, for example, by transforming with pJRDEE32d13 expressing the wild type phaC gene from Aeromonas caviae (Fukui et al., J. Bacteriol. 179:4821-4830, 1997; and U.S. Pat. No. 5,981,257).

By way of nonlimiting example with respect to, e.g., a phosphorus-deregulated bacteria, such a bacteria may have a mutation in a gene or a regulatory region (e.g. promoter, operator, DNA binding site for regulators, etc.) involved in phosphorus or phosphate detection, such that the bacteria appears to detect phosphorus depletion when there is none; or in a gene or regulatory region involved in phosphate transport; or in a gene or regulatory region of a positive phosphate regulator that activates other genes in response to phosphorus depletion (or conversely another regulator that represses genes in response to phosphorus depletion); or in a gene or regulatory region of a negative phosphate regulator that represses genes in response to excess phosphate (or conversely another regulator that activates genes in response to excess phosphate); or in one of the downstream genes (or regulatory regions thereof) whose transcription is regulated by such positive or negative phosphate regulators; or in one of the genes (or regulatory regions thereof) that modifies or regulates the primary positive and negative phosphate regulators. There can be different regulatory pathways under anaerobic and aerobic conditions.

Exemplary genes involved in phosphorus regulation include genes homologous to the E. coli genes pstA (phoT) (involved in phosphate transport), pstB (involved in phosphate transport), phoW (pstC) (involved in phosphate transport), phoS (phosphate binding protein), phoU (involved in phosphate transport), phoE (outer membrane porin), ugpB (glycerol-3-phosphate binding protein), phoR (negative phosphate regulator), phoB (positive phosphate regulator), phoM (positive phosphate regulator), psiE (phosphate starvation inducible gene), phn (psiD) (phosphate starvation inducible gene), phoG (psiH) (phosphate starvation inducible gene). See the description of such genes and pathways in, e.g., Amemura et al., J. Mol. Biol. 184:241-250, 1984; Makino et al., J. Mol. Boil. 190: 37-44, 1986; Wanner et al., “Phosphate Regulation of gene expression in Escherichia coli,” Escherichia coli and Salmonella typhimurium 1326-1333, 1987; Yamada et al., J. Bacteriol. 171:5601-5606, 1989; Rao et al., J. Bacteriol. 180: 2186-2193, 1998; Novak et al., J. Bacteriol. 181: 1126-1133, 1999; and Kim et al., J. Bacteriol. 182: 5596-5599, 2000.

Exemplary genes involved in nitrogen regulation include AmtA (involved in ammonium transport), GlnD (uridylyltransferase/uridylyl-removing enzyme) (involved in sensing intracellular nitrogen status), GlnB (P11) (involved in sensing intracellular nitrogen status), GlnK (signal transduction protein involved in regulation of nitrogen), glnE (adeylyltransferase), NtrB (sensory histidine protein kinase that is a nitrogen regulator), NtrC (DNA binding protein that is a nitrogen response regulator), rpoN (in some Pseudomonas species), GlnR (negative nitrogen regulator in Bacillus activated by excess nitrogen), TnrA (regulator in Bacillus activated by nitrogen limitation), CodY (negative regulator in Bacillus activated by excess nitrogen). Genes that are transcriptionally regulated by NtrBC include glnALG, glnHPQ, argT, hisJQMP, nasFEDCBA, nac, gltF. See the description of such genes and pathways in, e.g., Merrick et al., Microbiol. Rev. 59: 604-622, 1995; Atkinson et al., Molecular Microbiol. 29: 431-437, 1998; Fisher, Molecular Microbiol. 32: 223-232, 1999; and Blauwkamp et al., Molecular Microbiol. 46: 203-214, 2002.

The genome of Ralstonia metallidurans CH34, formerly Ralstonia eutropha and Alcaligenes eutrophus (and which has also been referred to as Cupriavidus necator or Wautersia eutropha) has been sequenced and the sequence data is made available by the Department of Energy's Joint Genome Institute (JGI). Genome sequence of Ralstonia eutrophus, formerly Alcaligenes eutrophus CH34, is also available from Brookhaven National Laboratory. Genome sequence of Ralstonia solanacearum is available from Genoscope and NCBI. The Ralstonia genome sequences may be searched, e.g. using BLAST, to identify sequences with homology to the regulators in E. coli or other bacteria.

The bacteria may be modified through application of radiation or other mutagenizing agents, or the bacteria may be modified via genetic engineering. Examples 1-3 illustrate the mutagenesis and selection of bacteria with the desired characteristics. Briefly, a PHA-negative mutant of R. eutropha is mutagenized and selected for phosphate deregulation, by growing the mutagenized bacteria in media containing non-limiting concentrations of phosphate and detecting alkaline phosphatase activity. Other nutrient-deregulated mutant bacteria are obtained using similar procedures (e.g., mutagenesis followed by growth in media containing non-limiting amounts of nitrogen, or other desired nutrient.)

Preferred mutant bacteria exhibit increased levels of constitutive production of PHA during the active multiplicative growth phase (also known as log phase), leading to near maximum accumulation of PHA at earlier time points even in the presence of non-limiting concentrations of nutrients, such as phosphate, nitrogen, etc.

Exemplary bacteria prepared in this manner have been deposited on Jun. 1, 2005 with the American Type Culture Collection (ATCC), P.O. Box 1549 Manassas, Va. 20108, USA, under Accession Nos. PTA-6759 and PTA-6760, respectively.

Descendants or mutants of such bacteria that retain same or similar mutations resulting in the desired deregulation characteristic(s) are contemplated by the invention. For example, such bacteria may be further mutagenized to isolate bacteria with added desirable characteristics, so long as they retain the characteristic of nutrient-deregulation. Additional desirable characteristics may also be conferred through recombinant genetic engineering. For example, new genes responsible for desired characteristics may be introduced via plasmids or recombination, or existing genes or regulatory regions in the bacteria's genome may be mutated to activate or inactivate them.

Moreover, the mutation(s) which are responsible for the observed phosphate- or nitrogen-deregulation phenotype can be identified, e.g., by sequencing the genome or gene products of the mutated bacteria to identify mutations, or restriction fragment length polymorphism analysis (digestion with restriction enzymes to identify different restriction sites created by the mutations), or hybridization pattern analysis (hybridization to probes of known sequence under hybridization conditions that differentiate between genome sequence that is identical to the probe(s) and genome sequence that contains mutations), or any other methods for mutation detection known in the art. Upon identification of the mutations, wild type bacteria may be genetically engineered through insertion or deletions in their genome to contain a mutation in the same region (e.g. regulatory region or gene).

If the bacteria are PHA-negative mutants, they are preferably additionally engineered to contain a desired PHA-producing gene, e.g. phaC, A and/or B, for example, by transforming with plasmids expressing phaC gene from Aeromonas caviae (Fukui et al., J. Bacteriol. 179:4821-4830, 1997; and U.S. Pat. No. 5,981,257), which enables PHA production. Alternative exemplary genes include phaC from Pseudomonas fluorescens (U.S. Pat. No. 6,475,734 or phaC from Aeromonas hydrophila 4AK4 (SEQ ID NO: 1). See U.S. Pat. Nos. 5,661,026 and 5,798,235.

Bacteria

This invention contemplates any bacteria capable of producing polyhydroxyalkanoate. These include, but are not limited to, species belonging to the genus Wautersia, Alcaligenes, Ralstonia, Zoogloea, Bacillus, Aeromonas, Azotobacter, Clostridum, Nocardia, Halobacterium and Pseudomonas. The bacteria may be native or genetically engineered. Among the above, Ralstonia, Bacillus, Pseudomonas, and Azotobacter are typically used. Wautersia eutropha, formerly known as Ralstonia eutropha, formerly referred to as Alcaligenes eutrophus, is most typically used. The bacteriological properties of these bacteria, belonging to the genus Ralstonia (Alcaligenes) are described in, for example, “BERGEY'S MANUAL OF DETERMINATIVE BACTERIOLOGY, Eighth Edition, The Williams & Wilkins Company/Baltimore”. The bacteria of the invention specifically exclude bacteria known to produce over 50% PHA by dry cell weight under culture conditions in which levels of nutrients are not limited, such as Alcaligenes latus and an Azotobacter vinlandii mutant (mutation in NADH oxidase), which each accumulate PHA to levels of over 90% of the weight of the cell.

Culture Medium

Essential nutrients required for growth of bacteria include a carbon source and at least the following inorganic elements, which are normally present in readily assimilable form, typically as water soluble salts: nitrogen, phosphorus, sulfur, potassium, sodium, magnesium, calcium, and iron, optionally with traces of manganese, zinc nickel, chromium, cobalt and/or copper.

The carbon sources are any substances which can be utilized by the bacteria, including synthetic, natural or modified natural carbon sources. Exemplary carbon sources include but are not limited to fatty acids, including hexanoic acid, heptanoic acid, octanoic acid, nonanoic acid, decanoic acid, and longer chain fatty acids; or the salt, ester (including a lactone in the case of a hydroxyl substituted acid), anhydride, amide or halide of the fatty acid; oils, including vegetable oil sources such as corn oil, soybean oil, palm kernel oil, cotton seed oil, rapeseed oil, peanut oil, fractionated oils of any of these types of vegetable oils, and/or derivatives thereof, and/or mixtures thereof; alcohols, including methanol, ethanol, hexanol, beptanol, octanol, nonanol and decanol as well as iso and other branched chain fatty acids or alcohols and acetic acid; other carbon sources such as carbon dioxide, yeast extract, molasses, peptone, and meat extract, saccharides such as arabinose, glucose, mannose, fructose, galactose, sorbitol, mannitol and inositol.

Exemplary nitrogen sources include inorganic nitrogen compounds such as ammonia, ammonium salts, nitrates, and/or organic nitrogen containing compounds such as urea, corn steep liquor, casein, peptone, yeast extract, and meat extract.

Exemplary inorganic components include calcium salts, magnesium salts, potassium salts, sodium salts, phosphoric acid salts, manganese salts, zinc salts, iron salts, copper salts, molybdenum salts, cobalt salts, nickel salts, chromium salts, boron compounds, or iodine compounds.

Optionally, vitamins can be included in the culture medium.

Bacterial Cultivation Methods

The nutrient-deregulated bacteria of the present invention can achieve good levels of PHA production even in media that is not limited in an essential nutrient. Methods of cultivating the bacteria simply include growing the bacteria in suitable culture media. The bacteria will produce significant levels of PHA even while multiplying. Any medium and appropriate culture conditions known in the art and/or described herein that foster multiplicative growth of the bacteria may be employed.

The culture temperature may vary depending on the organism. For Ralstonia, exemplary culture temperature may range from about 20 to 40° C., preferably about 25 to 35° C., and the pH is, for example, about 6 to 10, preferably about 6.5 to 9.5. The cultivation is carried out aerobically under these conditions.

Using rich media that allow for rapid growth and production of PHA, continuous fermentation reactors can be designed which could significantly lower the cost of production. (See Principles of Fermentation Technology, 2nd Edition, P. F. Stanbury, A. Whitaker and S. J. Hall, eds., Butterworth-Heinemann, publishers 1984, pp. 16-27; Henderson et al., Microbiology 143: 2361-2371, 1997; Ackermann et al., Polymer Degradation and Stability 59: 183-186, 1998.)

By way of example, a one-step cultivation process includes transferring the bacteria from one fermentor to another (scaling up), as well as batchwise or continuous feeding of culture medium. The lack of a second cultivation step in which one or more essential nutrients is restricted provides faster growth of bacteria, higher production of PHA, and a more industrially robust PHA production process at a reduced cost.

Two-Step Cultivation

Alternatively, the bacteria may be cultivated according to two-step cultivation methods, including conventional methods as described in, for example, U.S. Pat. No. 5,364,778 or U.S. Pat. No. 5,871,980 or U.S. Pat. No. 6,225,438. While it is not necessary to utilize such two-step cultivation methods for the bacteria of the present invention, the bacteria of the invention may be cultured according to such conventional methods.

Briefly, in the first stage the bacteria is grown under non-growth limiting conditions, and the bacteria is allowed to multiply until it reaches sufficient biomass, usually measured as a certain dry cell weight per liter. In the second stage, at least one nutrient required for growth is limited, such that multiplicative growth ceases and increased PHA production begins. While it is possible to enhance PHA production by restricting oxygen supply, the most practical nutrients to limit are nitrogen, phosphorus or less preferably, magnesium, sulfur, potassium, or iron.

Any carbon source can be used in either stage. In some prior art processes, the culture medium used in the first stage contains a readily metabolizable carbon source, such as a carbohydrate, for example, glucose, while the culture medium used in the second stage contains a more complex carbon source, for example a fatty acid or fatty alcohol.

In some processes, the bacteria cells are recovered by separation, by a conventional solid-liquid separation means such as filtration or centrifugation, from the culture broth obtained in the first step, and the cells thus obtained are subjected to cultivation in the second step. Alternatively, in the cultivation of the first step, a critical nutrient is substantially depleted and the culture broth can be migrated to cultivation in the second step without a recovery by separation of the cells to be cultured therein.

In one exemplary method, the bacteria are initially placed in culture media containing a sufficient supply of all nutrients to permit multiplicative growth. As the bacteria grow and consume nutrients, the supply of at least one essential nutrient, e.g. an inorganic element, is reduced to a limiting level that causes cessation of multiplicative growth and increased production of PHA. If culture media containing nutrients is resupplied to the bacteria, such culture media continues to be limited in at least this essential nutrient.

In an alternative exemplary method, in which culture media is supplied continuously or at various time intervals, the initial culture media supplied contains non-limiting levels of nutrients, and after a time period of sufficient multiplicative growth, the culture media supplied is limited in at least one essential nutrient.

U.S. Pat. No. 6,225,438 describes additional methods of culturing PHA-producing bacteria so that they produce PHA copolymers that have contain higher levels of medium chain length monomers (e.g. monomer units with greater than five carbons), resulting in a copolymer with increased flexibility and processing ability, reduced thermal decomposition during molding and excellent moldability, preferably with melting point temperatures of about 30 to 150° C. The methods involve culturing the bacteria in the presence of fatty acids and/or fatty alcohols with carbon chains containing six or more carbons and a fatty acid oxidation inhibitor. Such methods can be used with the bacteria of the present invention.

The carbon source and the fatty acid oxidation inhibitor may be added at any time during the cultivation in the second step from the initial stage to the end stage of cultivation. Addition at the initial stage is preferable. Examples of suitable fatty acid oxidation inhibitors according to this method include but are not limited to: acrylic acid, 2-butynoic acid, 2-octynoic acid, S-phenylproprionic acid, R-phenylproprionic acid, propiolic acid, and trans-cinnamic acid. The inhibitor can be the acid itself or a salt thereof. Sodium acrylate is one preferred fatty acid oxidation inhibitor. The fatty acid oxidation inhibitor may be used in an amount which can increase the accumulation of 3-hydroxyhexanoate (HH) (C6), 3-hydroxyheptanoate (HHp) (C7) and/or 3-hydroxyoctanoate (HO) (C8) copolymers by the desired amount but has an acceptable level of toxicity to the cells. For example, sodium acrylate may be used at a concentration in the culture medium of about 1-40 mM, preferably about 10-35 mM and more preferably 25-32 mM.

Enhancement of PHA Production by Nutrient Limitation

The bacteria of the present invention produce significant levels of PHA constitutively without the need to limit any essential nutrients during culture. However, even higher levels of PHA production may be achieved by nutrient limitation, for example, limitation of phosphorus, nitrogen, magnesium, sulfate, potassium or iron. Preferably, the limited nutrient is a nutrient for which the bacteria are not deregulated. For example, PHA production from one phosphate-deregulated organism described herein is not enhanced by phosphate limitation, but is enhanced by iron limitation. Production of PHA may be maximized by limiting various combinations of iron and another inorganic element.

In one embodiment, the iron concentration in the medium is limited such that the ratio of phosphate to iron is about 50 or higher, or about 350 or higher, or about 500 or higher, or about 2800 or lower, or about 1400 or lower or about 900 or lower. Exemplary ranges include from about 58 to about 2773, or about 350 to about 1400, or about 500-900 or about 600-800, e.g. 693.

Extraction of PHA Copolymer

The bacteria may be harvested from the culture broth by any means known in the art, including but not limited to conventional solid-liquid separation means such as filtration or centrifugation.

PHA copolymer may be extracted from the bacteria by any methods known in the art, including the following exemplary procedure.

The bacterial cells are harvested from the culture broth and the cells are washed once with 0.1M NaCl, 50 mM Tris-HCl, pH 8.0, suspended in water, then freeze dried. PHA copolymer is extracted into chloroform by refluxing for at least about five hours in about 50:1 chloroform to cell dry weight. The extract is filtered through Whatman #4 filter paper, dried down to a minimal volume, and the PHA copolymer is precipitated by adding the viscous solution to 10× volume of diethyl ether/hexane 3/1 v/v. The material is centrifuged in capped Teflon centrifuge tubes and washed once with ethyl ether/hexane before drying under vacuum overnight. Further fractionation of the PHA copolymer is performed by refluxing the solid ethyl ether/hexane precipitated PHA copolymer in boiling acetone for 5 hours. The acetone extract is dried under nitrogen, the PHA copolymer is dissolved into chloroform, and is precipitated with ethyl ether/hexane. Alternatively, the dried cells are directly extracted with acetone and the soluble PHA copolymer is isolated by drying down under nitrogen, dissolving into chloroform, and precipitating with ethyl ether/hexane.

Furthermore, the copolymer product can be recovered from the bacteria using various published procedures to produce PHA copolymer in a variety of useful physical forms. These include chemical extraction using chlorinated solvents (e.g., U.S. Pat. No. 4,562,245), non-chlorinated solvents (WO Publication No. 97/07230), marginal solvents (U.S. Pat. No. 5,821,299), the use of heat and enzymes for isolating PHA particles, an example of which is described in U.S. Pat. No. 4,910,145, or the use of physical means such as air classification (U.S. Pat. No. 5,849,854) and centrifugation (U.S. Pat. No. 5,899,339).

Quantification or Comparative Evaluation of PHA Production

Harvested cells are dried, weighed and the PHA extracted using chlorinated solvents as described. The extracted PHA is dried under vacuum overnight and weighed to obtain the percent PHA in the dried biomass and the titer in the fermented broth. Alternatively, PHA is quantitatively and qualitatively analyzed by gas chromatography (GC). Liquid cultures are centrifuged at 10,000 g for 15 minutes, and then the cells are washed twice in 0.9% sodium chloride saline and lyophilized overnight. Dried lyophilized cell material (8-10 mg) is subjected to methanolysis in the presence of 15% (v/v) sulfuric acid. The resulting methyl esters of the constituent 3-hydroxyalkanoic acids are assayed by GC according to Brandl et. al. (Appl. Environ. Microbiol. 54: 1977, 1988) and as described in detail (Timm et al., Appl. Environ. Microbiol. 56: 3360, 1990). GC analysis is performed by injecting 3 μL of sample into an Agilent Technologies model 6850 gas chromatograph (Waldbronn, Germany) using a 0.5 μm diameter Permphase PEG 25 Mx capillary column 60 m in length.

Uses of PHA Produced

The invention also provides PHA produced by the bacteria of the invention. Such PHA can be converted into fibers, molded articles, and film, and used in any applications known in the art for PHA or other plastics, including for medical materials such as surgical thread or bone setting materials, hygienic articles such as diapers or sanitary articles, agricultural or horticultural materials such as multi films, slow release chemicals, or fishery materials such as fishing nets, and/or packaging materials such as bottles, fast food wraps and boxes.

### EXAMPLES

The present invention is described in more detail with reference to the following non-limiting examples, which are offered to more fully illustrate the invention, but are not to be construed as limiting the scope thereof. The examples illustrate the isolation of nutrient-deregulated Ralstonia eutropha, the fermentation process of said deregulated Ralstonia eutropha for the production of PHA, and improved production of PHA by limiting trace elements.

### Example 1

### Isolation of a Phosphate-Deregulated Ralstonia eutropha

Phosphate-deregulated Ralstonia eutropha are isolated as follows. A PHA negative mutant of Ralstonia eutropha is cultured in a 100 mL shake flask containing yeast extract (10 mL, 3%) at 200 rpm for 16-20 hours at 30° C. until the OD₆₀₀ is over 10. The culture (3 ml) is transferred into 27 mL of sterile phosphate buffered saline (PBS). Five mL of diluted cells are removed for plating as an unmutated control, and the remaining suspension (25 mL) is exposed, with continuous stirring in the dark, to sufficient UV irradiation to give a survival rate of between 1 and 10%. A 1 mL aliquot of the irradiated culture is transferred to 11 mL of pre-warmed nutrient rich medium (1% w/v polypeptone, 1% w/v yeast extract, 0.5% w/v beef extract, 0.5% w/v ammonium sulphate, pH7) in a 100 mL shake flask. The flask is wrapped in aluminum foil to minimize photorepair and shaken in the dark at 200 rpm for 3 hours at 30° C. in order to allow segregation of the cells and “fixing” of mutations. The segregated cells are cultured on LB agar (1% w/v tryptone, 0.5% w/v yeast extract, 0.5% w/v sodium chloride, pH 7) containing the colorimetric alkaline phosphatase substrate, BCIP (40 μg/ml), and the cells are incubated for 2 to 3 days at 30° C. Intensely dark blue colonies are isolated as putative phosphate-deregulated strains of Ralstonia eutropha. Exemplary phosphate-deregulated Ralstonia eutropha are those deposited on Jun. 1, 2005 with the American Type Culture Collection (ATCC), P.O. Box 1549 Manassas, Va. 20108, USA, under Accession No. PTA-6759.

### Example 2

### Isolation of a Nitrogen-Deregulated Ralstonia eutropha

Nitrogen-deregulated Ralstonia eutropha are isolated as follows. A PHA negative mutant of Ralstonia eutropha is cultured in a 100 mL shake flask containing yeast extract (10 mL, 3%) at 200 rpm for 16-20 hours at 30° C. until the OD₆₀₀ is over 10. The culture (3 ml) is transferred to 27 mL of sterile phosphate buffered saline. Five mL of diluted cells are removed for plating as an unmutated control and the remaining suspension (25 ml) is exposed, with continuous stirring in the dark, to sufficient UV irradiation to give a survival rate of between 1 and 10%. A 1 mL aliquot of the irradiated culture is transferred to 11 mL of pre-warmed nutrient rich medium (1% w/v polypeptone, 1% w/v yeast extract, 0.5% w/v beef extract, 0.5% w/v ammonium sulphate, pH7) in a 100 mL shake flask. The flask is wrapped in aluminum foil to minimize photorepair and shaken in the dark at 200 rpm for 3 hours at 30° C. in order to allow segregation of the cells and “fixing” of mutations. The segregated cells are cultured on a minimal agar containing 200 mM of the ammonium analogue methylamine and a complex nitrogen source such as 0.1% w/v glycine and incubated for at least 3 days at 30° C. Colonies that appear to grow well are isolated as putative nitrogen-deregulated strains.

### Example 3

### Isolation of a Double-Deregulated (Both Phosphate- and Nitrogen-Deregulated) Ralstonia eutropha

Ralstonia eutropha, deregulated in both phosphate and nitrogen, are isolated as follows. A Ralstonia eutropha DSM541 phosphate-deregulated mutant from Example 1 is cultured in a 100 mL shake flask containing 10 mL 3% yeast extract at 200 rpm for 16-20 hours at 30° C. until the OD₆₀₀ is over 10. Three mL of the culture is transferred to 27 mL of sterile phosphate buffered saline. Five mL of diluted cells are removed for plating as an unmutated control and the remaining suspension (25 ml) is exposed, with continuous stirring in the dark, to sufficient UV irradiation to give a survival rate of between 1 and 10%. A 1 mL aliquot of the irradiated culture is transferred to 11 mL of pre-warmed nutrient rich medium (1% w/v polypeptone, 1% w/v yeast extract, 0.5% w/v beef extract, 0.5% w/v ammonium sulphate, pH7) in a 100 mL shake flask. The flask is wrapped in aluminum foil to minimize photorepair and shaken in the dark at 200 rpm for 3 hours at 30° C. in order to allow segregation of the cells and ‘fixing’ of mutations. The segregated cells are cultured on a minimal agar containing 200 mM of the ammonium analogue methylamine and a complex nitrogen source such as 0.1% w/v glycine and incubated for at least 3 days at 30° C. Colonies that appear to grow well are isolated as putative double-deregulated (both phosphate- and nitrogen-deregulated) strains. Exemplary phosphate- and nitrogen-deregulated Ralstonia eutropha are those deposited on Jun. 1, 2005 with the American Type Culture Collection (ATCC), P.O. Box 1549 Manassas, Va. 20108, USA, under Accession No. PTA-6760.

### Example 4

### Transformation of Nutrient-Deregulated Ralstonia eutropha with PHA Genes

Nutrient-deregulated Ralstonia eutropha cells are transformed with various PHA producing genes to enable PHA production, for example, by transforming with a plasmid pJRDEE32d13 expressing the wild type phaC gene from Aeromonas caviae (Fukui et al., J. Bacteriol. 179:4821-4830, 1997; and U.S. Pat. No. 5,981,257). A variety of transformation methods are known in the art, e.g., electroporation as described by Park et. al. (Biotechnology Techniques 9:31-34, 1995) or through transconjugation with E. coli S 17-1 as described by Friedrich et. al. (J. Bacteriology 147:198-205, 1981).

The bacteria are then grown in media containing a non-limiting concentration of deregulated nutrient, e.g., phosphorus, (or double deregulated nutrients, e.g., phosphorus and nitrogen) and measured for PHA accumulation as set out below.

### Example 5

### Measurement of PHA Accumulation

Bacteria are centrifuged, washed once with 0.1M NaCl, 50 mM Tris 8.0, centrifuged, suspended in 2-3 mL of water, frozen and lyophilized for 2 days. The dried cells are reacted in 1 mL chloroform plus 1 mL 15% sulfuric acid in methanol for 4 h at 100° C. Samples are phase separated with the addition of 1 mL of chloroform plus 1 mL of 1M NaCl. The chloroform phase is treated with anhydrous sodium sulfate to dry, 1 mL is removed and dried in a sample vial under nitrogen or overnight in the hood. Samples are dissolved in 1 mL of acetone plus 1 g/L methyl benzoate and capped. Alternatively, 100 μL of 10 g/L methylbenzoate is added directly to the 1 mL of chloroform; and the samples are capped and analyzed.

Samples are analyzed on a HP5890 GC using a 30 m, 0.32 mmID, 0.25 μm film Supelcowax 10 column using helium at a 1 cm³/sec flow rate equal to 20 cm/sec linear flow rate. The injector is set at 225° C. and FID detector at 300° C. The oven temperature is kept at 80° C. for 2 min following a 1 μL injection (50:1 split), ramped at 10° C./min to 230° C., and kept at 230° C. for 12 min. Alternatively, samples are analyzed on a J&W DB-5MS (part 122-5531, 30 m, 0.25 mm ID, 0.1 um film) using the same program as above, but the final temperature is kept at 230° C. for 7 min.

3-hydroxyalkanoates and methyl 3-hydroxyalkanoates, for use as standards, are purchased from Sigma (catalog #H6501 dl-beta-hydoxybutyric acid, catalog # H4023 3-hydroxycaprylic acid methyl ester, catalog # H3773 3-hydroxycapric acid methyl ester, catalog # H3523 3-hydroxylauric acid methyl ester, catalog # H4273 3-hydroxymyristic acid methyl ester, catalog # H4523 3-hydroxypalmitic acid methyl ester) and 3-hydroxycaproic acid methyl ester is identified from a PHA copolymer processed from Aeromonas hydrophila grown on lauric acid.

### Example 6

### Fermentation of a Phosphate-Deregulated Ralstonia eutropha for the Production of Poly-3-Hydroxylalkanoate (PHA)

The fed batch culture of a recombinant phosphate-deregulated Ralstonia eutropha, engineered to be capable of producing polyhydroxybutyrate-co-hexanoate, is carried out using vegetable oil, fatty acids, fatty alcohols and esters as the feed substrate. Vegetable oils include: corn oil, soybean oil, palm oil, palm kernel oil, cotton seed oil, rapeseed oil, peanut oil, their fractionated oil, their mixture. The fed batch culture can also make PHA-utilizing carbohydrate feeds including fructose, gluconic acid, glucose, and molasses, some of which require a strain selected for growth on the particular feed.

The strain is first grown at 30° C. in 100 mL of nutrient broth to an OD₆₀₀ of 2.0. This broth is then used to inoculate and grow 3 L of a seed culture fermenter using the following medium:

Seed Fermenter:

| | | |
|---|---|---|
| Na₂HPO₄ | 11.0 | g/L |
| KH₂PO₄ | 1.90 | g/L |
| (NH₄)₂SO₄ | 12.87 | g/L |
| MgSO₄•7H₂O (20 g/100 ml) | 5 | ml/L |
| Trace Elements | 5 | mL/L |
| CoCl₂•6H₂O | 0.218 | g |
| FeCl₃•6H₂O | 16.2 | g |
| CaCl₂•2H₂O | 10.3 | g |
| NiCl₂•6H₂O | 0.118 | g |
| CuSO₄•5H₂O | 0.156 | g |

Dilute to 1 L with 0.1N HCl

Sterilize medium at 121° C. for 20 min; cool, and add 30 g/L vegetable oil and 50 mg/L kanamycin.

Operate at:

| | |
|---|---|
| Temperature | 30° C. |
| Initial pH | 6.8 |
| pH control point | 6.8 with 7% NH₄OH |
| Aeration | 0.6 vvm |
| Agitation | 500 rpm |

The culture is harvested when the OD₆₀₀=69.9, and 175 mL of the culture is used to inoculate 3.5 L in the main fermentors. The concentration of phosphate in the medium is measured using a Nova Biomedical 300 Bioprofile Analyzer and is shown never to be limiting.

| | | |
|---|---|---|
| Na₂HPO₄ | 4.36 | g/L |
| KH₂PO₄ | 1.90 | g/L |
| (NH₄)₂SO₄ | 2.91 | g/L |
| antifoam | 3 | ml/L |
| MgSO₄•7H₂O (20 g/100 mL) | 5 | mL/L |
| Trace Elements | 5 | mL/L |

Operate at:

| | |
|---|---|
| Temperature | 28° C. |
| Initial pH | 6.8 |
| pH control point | 6.8 with 14% NH₄OH |
| Aeration | 0.6 vvm |
| Agitation | 400 rpm |
| Back Pressure | 1-2 psi |

Feed with a total of 110 g/L vegetable oil at:

Time

| | | |
|---|---|---|
| 0 | h | 0.56 ml/L |
| 2 | h | 0.92 |
| 4 | h | 1.28 |
| 6 | h | 1.67 |
| 8 | h | 2.02 |
| 10 | h | 2.37 |
| 12-60 | h | 2.67 |

The strain is also grown on phosphate limiting medium in which the complete utilization of added phosphorous during the culture, usually between 20 h and 36 h culture time, induces wild type strains to increase production of PHA.

Limiting Phosphate Medium:

| | | |
|---|---|---|
| Na₂HPO₄ | 3.85 | g/L |
| KH₂PO₄ | 0.67 | g/L |
| (NH₄)₂SO₄ | 2.91 | g/L |
| antifoam | 3 | mL/L |
| MgSO₄•7H₂O (20 g/100 ml) | 5 | mL/L |
| Trace Elements | 5 | mL/L |

Inoculate with 5% v/v of seed fermenter

Operate at:

| | |
|---|---|
| Temperature | 30-34° C. for 16 h, then 28° C. |
| Initial pH | 6.8 |
| pH control point | 6.8 with 14% NH₄OH |
| Aeration | 0.6 vvm |
| Agitation | 400 rpm |
| Back Pressure | 1-2 psi |

Feed with a total of 110 g/L vegetable oil at:

Time

| | | |
|---|---|---|
| 0 | h | 0.56 ml/L |
| 2 | h | 0.92 |
| 4 | h | 1.28 |
| 6 | h | 1.67 |
| 8 | h | 2.02 |
| 10 | h | 2.37 |
| 12-60 | h | 2.67 |

The richer medium allows the cells to grow with minimal lag time. Cells grown at a higher phosphate concentration are constitutive for PHA production and accumulate PHA at all times of cell growth. Cells grown at a higher phosphate concentration produce PHA at 165% of the amount of PHA produced by cells grown on limiting phosphate. By comparison, a PHA producing R. eutropha with a non-deregulated background produces only 55% of the PHA in the high phosphate medium as in the limiting phosphate medium.

This is the first time that significant levels of constitutive production of PHA during the active growing phase (log phase) of R. eutropha is demonstrated, leading to near maximum incorporation of PHA at earlier time points, regardless of the presence of non-limiting concentrations of phosphate.

### Example 7

### Fermentation of a Nitrogen-Deregulated, Double-Deregulated, and other Nutrient-Deregulated Ralstonia eutropha for the Production of PHA

Nitrogen-deregulated strains, double deregulated strains (both phosphate- and nitrogen-deregulated), and other nutrient-deregulated strains of R. eutropha are grown in a non-limiting nutrient broth as described in Example 6 to produce PHA at greater concentrations than native R. eutropha.

### Example 8

### Fermentation of a Nutrient-Deregulated Ralstonia eutropha in Rich Media for the Production of PHA

Nutrient-deregulated strains do not require induction by limiting nutrients to make large quantities of PHA. Therefore, complex media that are rich sources of nutrients may be added to increase overall biomass production while retaining high PHA production, thus, increasing the overall titer of PHA in a given culture vessel. Rich media may include yeast extract, peptone, tryptone, amino acids, beef extract and other sources of organic nitrogen, phosphate and other nutrients.

### Example 9

### Fermentation of a Nutrient-Deregulated Ralstonia eutropha in Richer Inexpensive Media for the Production of PHA

Nutrient-deregulated strains do not require induction by limiting nutrients to make large quantities of PHA; however, adding expensive rich sources of media still may not be economical. Nutrients are supplemented more economically by supplying e.g., phosphoric acid, ammonium salts, nitrates, nitrites, corn steep liquor, soybean hydrolysate, crude glycerin, whey, industrial process oil, carbohydrate or protein waste streams, or other less expensive simple or complex media without concern of inhibiting PHA production due to supplying a critical nutrient in excess.

### Example 10

### Induction of High Levels of PHA Production in Nutrient-Deregulated Ralstonia eutropha by Limiting Trace Elements

Phosphate-deregulated and nitrogen-deregulated strains of R. eutropha are constitutive producers of PHA in non-limiting phosphorous and nitrogen media, respectively. By limiting trace elements in the culture media, while feeding vegetable oil, PHA production increases.

Ralstonia eutropha, nutrient-deregulated in phosphate or in phosphate and nitrogen, and capable of producing polyhydroxybutyrate-co-hexanoate (C4C6), are grown in media containing the following:

| | | |
|---|---|---|
| Na₂HPO₄ | 7.70 | g/L |
| KH₂PO₄ | 1.34 | g/L |
| (NH₄)₂SO₄ | 2.91 | g/L |
| MgSO₄•7H₂O (20 g/100 mL) | 5 | mL/L |
| Antifoam added as needed | | |
| Trace Elements | 0-1 | mL/L |
| CoCl₂•6H₂O | 0.218 | g |
| FeCl₃•6H₂O | 16.2 | g |
| CaCl₂•2H₂O | 10.3 | g |
| NiCl₂•6H₂O | 0.118 | g |
| CuSO₄•5H₂O | 0.156 | g |

Dilute to 1 L with 0.1N HCl

The bacteria are fed batchwise with vegetable oil. Typical levels are 5 mL/L Trace Elements, 3.85 g/L Na₂HPO₄ and 0.67 g/L KH₂PO₄.

Levels of PHA over 85% by weight of the cell are observed. In comparison, phosphate deregulated cells accumulate 35% PHA in phosphate limited medium, and 49% PHA in non-limiting medium. Non-deregulated cells do not accumulate PHA in non-limiting medium and accumulate only 1.9% PHA in trace element limiting medium.

In addition, for the phosphate-deregulated mutant expressing the wild type phaC gene (pJRDEE32d13) from Aeromonas caviae (Fukui et al., J. Bacteriol. 179:4821-4830, 1997; and U.S. Pat. No. 5,981,257), limiting trace elements in the medium by 80% improves PHA production by 74% PHA in comparison with PHA production of 35% PHA under phosphate-limited conditions in the same amount of biomass.

R. eutropha (described in Example 1), transformed with a plasmid containing the Aeromonoas caviae phaC gene, are fermented in 3.5 L starting volume using a control medium in which 2× phosphate salts are added, and a test medium in which the amount of trace elements is adjusted. The bacteria are fed corn oil and acid is neutralized with ammonium hydroxide. Restriction of trace elements in the test medium results in doubling of the PHA content of the cells compared to the control culture medium. Restricting trace element quantities to 20% of the amount in the control medium is sufficient to support cell growth equivalent to growth on control medium while still providing increased PHA content of the cells.

Table 1 below shows the effect of limiting trace elements on phosphate deregulated cell growth and PHA accumulation vs. trace elements. When cultured under phosphate limited conditions, the cells contain 35% by weight of PHA (weight of PHA per dry cell weight (DCW)), have a productivity of 0.51 gPHA/L/h, and produce 39.3 g/L PHA. Severely limiting trace elements by 90% or more, while supplying excess phosphate, enables high PHA accumulation per cell, but limits cell growth and PHA production. Depleting the trace elements by 80% allows for full cell growth and 84.1 g/L PHA production (73.6% PHA by weight and productivity of 1.17 gPHA/L/h).

| | pJRDEE32d13 in Phosphate-Deregulated Mutant PGC-5 | | | | | |
|---|---|---|---|---|---|---|
| | Phosphate | | | | | |
| | Limiting | 0 Trace | 2% Trace | 5% Trace | 10% Trace | 20% Trace |
| | Media | Elements | Elements | Elements | Elements | Elements |
| DCW g/L | 106 | 61 | 70 | 67 | 76 | 114 |
| % PHA | 35 | 73 | 72 | 65 | 68 | 73.6 |
| g PHA/L/h | 0.51 | 0.62 | 0.7 | 0.6 | 0.72 | 1.17 |
| Yield g PHA/g | 0.35 | 0.38 | 0.42 | 0.38 | 0.45 | 0.76 |
| Oil | | | | | | |
| g PHA/L | 39.3 | 51.5 | 56.1 | 50.5 | 58.5 | 84.1 |

### Example 11

### Improved PHA Production in Nutrient-Deregulated Ralstonia eutropha by Limiting Iron

In further testing of varying quantities of trace elements, culture medium containing 20% of the iron present in the control medium, but the full amounts of other trace elements, yields the same results. These results indicate that iron restriction provides the desired effect of increasing PHA production.

An exemplary phosphate to iron ratio (molarity/molarity) used in culture, which demonstrates increased PHA accumulation, is set out below. Fermentation media demonstrating improved PHA accumulation includes phosphate (41.6 mM) and iron (0.06 mM) for a ratio of 693:1. It is also possible to decrease the iron concentration by half or double the phosphate for a ratio of 1,387:1. Ratios of phosphate to iron may vary from about 58:1 to about 2773:1. The exemplary ratios of P to Fe are not meant to be limiting. One of skill in the art can determine such ratios.

Throughout this application, various publications are referenced. The disclosures of these publications in their entirety, including but not limited to the specific aspects of the disclosures referenced herein, are hereby incorporated by reference into this application in order to more fully describe the state of the art as known to those skilled therein as of the date of the invention described and claimed herein.

The disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. Isolated nutrient-deregulated bacteria capable of (a) producing at least 10% polyhydroxyalkanoate (PHA) by dry cell weight under culture conditions in which levels of nutrients are not limited and (b) producing at least 20% PHA by dry cell weight under culture conditions in which iron is limited but no other nutrients are significantly limited, wherein said bacteria exclude Alcaligenes latus and an Azotobacter vinlandii mutant which has a mutation in NADH oxidase.

2. Isolated bacteria that exhibit all of the identifying characteristics of the bacteria deposited under ATCC Accession No. PTA-6759.

3. Isolated bacteria that exhibit all of the identifying characteristics of the bacteria deposited under ATCC Accession No. PTA-6760.

4. The bacteria of claim 1 which are a Wautersia, Ralstonia, Bacillus, Nocardia, Aeromonas, or Pseudomonas species.

5. The bacteria of claim 1 which are a phosphate-deregulated Wautersia species.

6. The bacteria of claim 2 which are phosphate-deregulated Wautersia species.

7. The bacteria of claim 1 which are phosphate- and nitrogen-deregulated.

8. The bacteria of claim 2 which are phosphate- and nitrogen-deregulated.

9. The bacteria of claim 1 which contain a non-native PHA-producing gene.

10. The bacteria of claim 2 which contain a non-native PHA-producing gene.

11. The bacteria of claim 3 which contain a non-native PHA-producing gene.

12. The bacteria of claim 9 wherein the PHA-producing gene is selected from phaA, phaB, or phaC genes.

13. A method of producing PHA using bacteria capable of (a) producing at least 10% polyhydroxyalkanoate (PHA) by dry cell weight under culture conditions in which levels of nutrients are not limited and (b) producing at least 20% PHA by dry cell weight under culture conditions in which iron is limited but no other nutrients are significantly limited, wherein said bacteria exclude Alcaligenes latus and an Azotobacter vinlandii mutant which has a mutation in NADH oxidase, said method comprising the step of growing said bacteria in culture media so that said bacteria produce PHA.

14. A method of producing PHA using bacteria that exhibit all of the identifying characteristics of the bacteria deposited under ATCC Accession No. PTA-6759, said method comprising the step of growing said bacteria in culture media so that said bacteria produce PHA.

15. A method of producing PHA using bacteria that exhibit all of the identifying characteristics of the bacteria deposited under ATCC Accession No. PTA-6760, said method comprising the step of growing said bacteria in culture media so that said bacteria produce PHA.

16. The method of claim 13 wherein said bacteria are cultured in media that is not limited in an essential nutrient.

17. The method of claim 16 further comprising the step of culturing the bacteria in culture media that is limited in one or more inorganic elements.

18. The method of claim 17 wherein the limited element is iron.

19. The method of claim 13 wherein the culture medium comprises a vegetable oil.

20. The method of claim 14 wherein the culture medium comprises a vegetable oil.
